(19) United States
(12) Patent Application Publication
(10) Pub. No.: US 2003/0077566 A1
(43) Pub. Date: Apr. 24, 2003

(54) Method for cryopreserving mammalian cells and tissues

(76) Inventors: Andrzej T. Palasz, Saskatoon (CA); Reuben J. Mapletoft, Saskatoon (CA); Heng Wang, Saskatoon (CA); Albert J. Robertson, Saskatoon (CA); Lawrence V. Gusta, Saskatoon (CA)

Correspondence Address: KIRBY EADES GALE BAKER, BOX 3432, STATION D, OTTAWA, ON, K1P 6N9, CA

(21) Appl. No.: 10/237,065

(22) Filed: Sep. 9, 2002

Related U.S. Application Data
(60) Provisional application No. 60/317,924, filed on Sep. 10, 2001.

(51) Int. Cl. A01N 1/02; C12N 5/06

(52) U.S. Cl. 435/2; 435/374

(57) ABSTRACT

Mammalian cells and tissues are cultured and preserved by mixing the cells or tissues with an extender derived from phosphatidyl-choline of plant origin, e.g. soybeans, and lowering the temperature of the mixture sufficiently to preserve the mammalian cells and tissues in viable form.

### CROSS-REFERENCE TO RELATED APPLICATION

This application claims the benefit of Provisional Application Serial No. 60/317,924, filed Sep. 10, 2001.

### FIELD OF THE INVENTION

The present invention relates to methods for the culture and low temperature preservation of mammalian cells and tissues, e.g. semen, and to methods for the recovery of mammalian cells and tissues which have been preserved at low temperatures.

### BACKGROUND OF THE INVENTION

Cryopreservation is based on the reduction and subsequent arrest of metabolic functions of biological materials stored at low temperatures. For cryopreserving mammalian cells and tissues, e.g. mammalian semen, it has long been known to combine the semen with an extender containing permeating cryoprotectant in a buffered solution prior to freezing. For example, Colas, U.S. Pat. No. 3,973,003, issued Aug. 3, 1976, describes diluting semen in a diluent consisting of milk or egg yolk and containing glycerol prior to cryopreservation. Another procedure for cryopreservation of semen that involves the use of egg yolk can be found in Aitken, U.S. Pat. No. 6,130,034, issued Oct. 10, 2000.

There is a concern about the use of additives of animal origin, such as egg yolk, for this purpose in that they may present a risk of pathogenic virus transmission.

In Stachecki, U.S. Pat. No. 5,985,538, there is described a method for cryopreservation of cells in which a choline salt is used.

It is also known to use hyaluronic acid as part of a culture solution and the cryopreservation solutions for cells and tissues. This is described, for instance, in Alkemade et al. U.S. Pat. No. 5,102,783, issued Apr. 7, 1992, where cells such as embryos, ova and sperm were cryopreserved. A purpose was to eliminate the risk of pathogen and virus transmission during the cryopreservation procedure.

It is an object of the present invention to find an extender of plant origin which will be at least as effective as egg yolk in the cryopreservation of mammalian cells and tissues, while avoiding the risk of disease transmission.

### SUMMARY OF THE INVENTION

This invention relates to a method for culturing and preserving mammalian cells and tissues in which the mammalian cells or tissues being preserved are mixed with an extender containing a lipid of plant origin, e.g. a phospholipid and thereafter lowering the temperature of the mixture sufficiently to preserve the mammalian cells and tissues in a viable condition.

The mammalian cells and tissues may include a wide range of materials, such as mammalian semen, oocytes, embryos, blood, etc. The method of the invention has been found to be particularly effective in the cryopreservation of bull semen.

Soybeans are an excellent source of phospholipids, particularly phosphatidyl-choline and, in terms of cost and effectiveness, phosphatidyl-choline of soybean origin has been found to be well suited for the purpose of the invention. The concentration of this phosphatidyl-choline is not critical for effective preservation, but is preferably present in the mixture in an amount of at least about 1 g/l of the mixture. For instance, it has been found to be very effective as an extender in the cryopreservation of bull semen when used a material containing about 5% to 50% by weight, preferably about 15%, of phosphatidyl-choline, at concentrations of about 0.20 g to 20 g, preferably about 0.730 g to 7.30 g, per 100 mls of mixture.

It has also been found to be particularly advantageous to use the phosphatidyl-choline in combination with glycoprotein-hyaluronan. This combination provides a further advantage over egg yolk as an extender in cryopreservation. When phosphatidyl-choline is used in combination with hyaluronan, e.g. in the cryopreservation of bull semen, there is significantly more oocyte cleaved and embryo developed to the hatched blastocyst stage than when the traditional egg yolk extender is used. The hyaluronan is preferably used in a ratio of hyaluronan to phospholipids of from about 0.25 mg/ml to 0.5 mg/ml per 7.30 g/100 ml of phospholipids.

Mammalian cells and tissues cryopreserved according to the method of this invention can be stored in chilled liquid form or in frozen solid form. The chilled liquid form is typically at a temperature of about +4° C., while the frozen solid form is at cryopreservation temperatures as low as −196° C. The chilled temperatures above freezing are typically used for storage periods of up to about 72 hours, while the cryopreservation temperatures are used for long term storage.

### DESCRIPTION OF THE PREFERRED EMBODIMENTS

### Example 1

Cryopreservatives of bull semen were carried out using three different extenders. These included phosphatidyl-choline and two previously known extenders for comparative studies. The three extenders were the following:

1. Phosphatidyl-choline

| | | |
|---|---|---|
| Tris (hydroxymethyl aminoethane) | 3.028 | g |
| Citric acid monohydrate | 1.675 | g |
| Fructose | 1.250 | g |
| Millipore water | 92.0 | ml |
| Glycerol | 8.0 | ml |
| Soybean phospholipids* | 1.46 | g |

*Lecithin isolated from soybean plants containing about 14.5% phosphatidyl-choline, obtained from Sigma Chemical Company.

2. Egg yolk

| | | |
|---|---|---|
| Triladyl** | 1.25 | ml |
| Water | 3.75 | ml |
| Egg yolk | 1.25 | ml |

**Triladyl is the commercial name for bull semen extender containing 7% glycerol.

3. Biociphos-plus (French made semen extender containing no egg yolk).

General Materials and Methods

The semen was collected by electro ejaculation from a 36-month-old Charolais bull twice a week. Aliquots of semen from each ejaculate were diluted with each of the extenders used to a concentration of 120×10⁶ spermatozoa per ml, cooled to 4° C. over 4 hour, aspirated into 0.5 ml plastic straws, frozen 4 cm above liquid nitrogen for 10 min and then plunge into liquid nitrogen. Frozen semen was thawed at 37° C. in a water bath for 20 sec, until content of the straw was melted. The effect of various semen extenders on the sperm motility was analyzed under microscope after each stage of cooling/freezing procedure. Sperm viability was tested within in vitro fertilization and artificial insemination trials. Parameters of motility of the raw semen were assessed and used as reference.

Individual Sperm Motility Assay

Observation of individual motility and estimation of the percentage of progressively motile cells provides information about sperm membrane integrity as well as the morphological integrity of spermatozoa. Motile spermatozoa are dependent upon pH, temperature and osmolarity. For this reason new, perfectly clean, warm slides and cover slips were always used. In order to evaluate individual motility a 10 μl drop of fresh semen was placed on warm slide and covered by the cover slip. Wet mounts were examined at 200 to 500×magnifications, under phase-contrast microscopy and 200 sperm were evaluated each time. After thawing, semen was kept in the incubator at 37° C.

The results are given in Table 1 below which shows the percent motility of the semen after dilution, cooling and freezing.

TABLE 1

| | Motility of Semen (%) | | |
|---|---|---|---|
| | After dilution | After 4 hr | After freezing |
| Extender | at 37° C. | at 4° C. | 0 hr |
| None (raw semen) | 85-90 | — | — |
| Triladyl + egg yolk | 90 | 70 | 55-60 |
| Biociphos plus | 90 | 60 | 35 |
| Phosphatidyl-choline | 90 | 75 | 35-40 |

The above results show that phospholipids containing 14.5% phosphatidyl-choline have the properties required for protecting bull semen during cooling-freezing procedures.

### Example 2

The procedure of Example 1 was repeated to determine the optimal concentration of lecithin (containing 14.5% phosphatidyl-choline) for the post-thaw survival of the bull semen. The semen from each ejaculate was diluted in the lecithin at four different concentrations and tested along with Triladyl+egg yolk as a control. The four concentrations of lecithin were 7.30 g, 1.46 g, 0.730 g and 0.365 g/100 ml. The osmolarity and pH of the extenders used were not affected by the different concentrations and remained at 320 mOsm and 7.12 respectively.

The results obtained are given in Table 2 below, where percentage motility of the semen is shown for each stage.

TABLE 2

| | Motility of Semen (%) | | |
|---|---|---|---|
| | After dilution | After 4 hr | After thawing |
| Extender | at 37° C. | at 4° C. | 1 hr |
| None (raw semen) | 80 | — | — |
| Triladyl + egg yolk | 85 | 80 | 55 |
| Lipids 7.30 g/100 ml | 85 | 80 | 50 |
| Lipids 1.46 g/100 ml | 85 | 85 | 45 |
| Lipids 0.730 g/100 ml | 85 | 85 | 40 |
| Lipids 0.365 g/100 ml | 85 | 85 | 40 |

The above table shows that there was no difference in post-thaw sperm viability between the control and the 7.30, 1.46 g and 0.730 g/100 ml phospholipids. However, there was a decreased sperm viability with the lowest (0.365 g/100 ml) concentration phospholipids as compared to the control group.

### Example 3

For this test different concentrations of a 40% preparation of phosphatidyl-choline of soybean origin obtained from Sigma Chemical Company were tested for the post-thaw survival of the bull semen.

Semen samples were collected from 5 bulls (3 Charlois, 2 Hereford) and each ejaculate was diluted in extenders containing different concentrations of a 40% phosphatidyl-choline preparation of soybean origin. Triladyl+egg yolk extender was used as a control.

The results are given in Table 3 below, where percentage motility of the semen is shown for each stage of the cryopreservation procedure and one hour after thawing.

TABLE 3

| | Motility of Semen (%) | | |
|---|---|---|---|
| | After dilution | After 4 hr | After thawing |
| Extender | at 37° C. | at 4° C. | 1 hr |
| None (raw semen) | 85 | — | — |
| Triladyl + egg yolk | 85 | 65 | 40-45 |
| Lipids 7.30 g/100 ml | 80 | 60 | 40 |
| Lipids 1.46 g/100 ml | 80 | 65 | 40-45 |
| Lipids 0.730 g/100 ml | 75-80 | 65 | 40-45 |
| Lipids 0.365 g/100 ml | 80 | 65 | 35-40 |

Similar post-thaw viabilities of sperm were obtained for extenders containing egg yolk and the three highest concentrations of the phospholipids. However, the lower phospholipid concentration of 0.365 g/100 ml showed a lower post-thaw motility.

### Example 4

This was a test to determine the effect of two different concentrations of a 14.5% and 40% phosphatidyl-choline preparations on the post-thaw survival of bull semen.

The semen from each ejaculate was diluted with Triladyl+egg yolk extender (control); and with 7.30 g/100 ml of 40% preparation; 0.365 g/100 ml of 40% preparation; 7.30 g/100 ml of 14% preparation and 0.365 g/100 ml of 14% preparation of phosphatidyl-choline. After dilution, semen from all experimental groups were cooled and frozen. Sperm individual motility was visually estimated by counting 10 microscopic fields after dilution, after 4 hours at 4° C. and immediately (0 hours) after thawing.

The osmolarity and pH of all the extenders used were not affected by the different concentrations of phospholipids used and were 320 mOsm and 7.12 respectively. The results are shown in Table 4 below.

TABLE 4

| | Motility of semen (%) | | |
|---|---|---|---|
| | After dilution | After 4 hr | After freezing |
| Extender | at 37° C. | at 4° C. | 0 hr |
| Triladyl + egg yolk | 85 | 80 | 50 |
| Lipids 7.30 g/40% | 80 | 70 | 45 |
| Lipids 0.365 g/40% | 80 | 60 | 25 |
| Lipids 7.30 g/14% | 85 | 80 | 60 |
| Lipids 0.365 g/14% | 85 | 70 | 21 |

It can be seen that there was considerably lower sperm viability with phospholipid concentrations of 0.365 g/100 ml.

### Example 5

The purpose of this test was to determine whether addition of soybean lipids to commercially used extender increases post-thaw viability of bull semen.

Semen was collected and processed before freezing in the manner described in previous examples and each ejaculate was divided into the four following groups: (1) Triladyl+egg yolk (control); (2) Triladyl+egg yolk+7.30 g/100 ml 40% lipids; (3) Triladyl+egg yolk+7.30 g/100 ml 14.5% lipids; (4) 50% of the above extender (1) and 50% of the above extender (3). As seen from Table 5 below, there was no effect of exogenous phospholipids on post-thaw semen viability.

TABLE 5

| | Motility of the semen (%) | | |
|---|---|---|---|
| | After dilution | After 2 hr | After freezing |
| Extender | at 37° C. | at 4° C. | 0 hr |
| Triladyl + egg yolk | 85 | 80 | 50 |
| (control) | | | |
| Lipids 7.30 g/100 ml | 80 | 70 | 55 |
| (40%) + egg yolk | | | |
| Lipids 7.30 g/100 ml | 80 (slow) | 65 | 60 |
| (14%) | | | |
| Control + 7.30 g/100 ml | 85 | 80 | 60 |
| (14%) | | | |
| 50% Group 1 + 50% of | 85 | 70 | 60 |
| Group 3 | | | |

### Example 6

The object of this test was to determine the effect of adding hyaluronan (MAP-5) to the bull semen extender containing phosphatidyl-choline on post-thaw semen viability.

Semen was collected and processed before freezing in the manner described in previous examples and aliquots of each ejaculate were diluted with (1) Triladyl+egg yolk (control); (2) soybean phospholipid (7.30 mg/100 ml)+1 mg/100 ml MAP-5; (3) soybean phospholipid (7.30 mg/100 ml)+0.5 mg/100 ml MAP-5; (4) soybean phospholipid (7.30 mg/100 ml)+0.25 mg/100 ml MAP-5.

Semen motility after dilution, cooling and freezing in the above extenders are shown in Table 6 below.

TABLE 6

| | Motility of the semen (%) | | |
|---|---|---|---|
| | After dilution | After 4 hr | After freezing |
| Extender | at 37° C. | at 4° C. | 0 hr |
| Triladyl + egg yolk | 80 | 70 | 57 |
| (control) | | | |
| Lipids 7.30 mg/100 ml + | 80 | 75 | 60 |
| 1 mg/ml MAP-5 | | | |
| Lipids 7.30 mg/100 ml | 80 | 70 | 56 |
| 0.5 mg/ml MAP-5 | | | |
| Lipids 7.30 mg/100 ml | 80 | 70 | 55-60 |
| 0.25 mg/ml MAP-5 | | | |

From the above results it can be seen that the addition of MAP-5 of any concentration had an effect on the post-thaw semen motility.

Example 7

The objective of this test was to compare the fertilizing 20 ability of semen frozen in extender containing egg yolk or phosphatidyl-choline of soybean origin+hyaluronan. The frozen semen was prepared in the same manner as in the above examples and was used for in vitro fertilization of bovine oocytes. The results are shown in Table 7 below.

TABLE 7

| | Total | Number | | |
|---|---|---|---|---|
| | oocytes | oocytes | Blastocysts (%) | |
| Extender | inseminated | cleaved (%) | Number | Hatched |
| Triladyl + egg yolk | 466 | 218 (46.8)^{a} | 37 (16.9) | 10 (27.0)^{a} |
| (control) | | | | |
| Lipids 7.30 mg/ | 458 | 281 (61.3)^{b} | 65 (23.1) | 29 (44.6)^{b} |
| 100 ml + 0.5 mg/ | | | | |
| 100 ml MAP-5 | | | | |

^{ab}-P < 0.05

The above results show that there was significantly more oocyte cleaved and embryo developed to the hatched blastocyst stage in the soybean phospholipid+hyaluronan extender as compared to the egg yolk extender.

### Example 8

Semen frozen in the same manner as in Example 6 was used for artificial insemination trials on 11 heifers. The results are shown in Table 8 below.

TABLE 8

| | | Total | | |
|---|---|---|---|---|
| | | embryos | Fertilized | Transferable |
| | Heifers | collected | embryos | embryos |
| Extender | inseminated | N (%) | N (%) | N (%) |
| Triladyl + egg yolk | 11 | 77 | 54 (70.1) | 35 (54.7) |
| (control) | | | | |
| Lipids 7.30 mg/ | 11 | 81 | 74 (91.4) | 55 (74.3) |
| 100 ml + 0.5 mg/ | | | | |
| 100 ml MAP-5 | | | | |

1. A method for culturing and preserving mammalian cells and tissues comprising the steps of:
(a) mixing mammalian cells or tissues with an extender comprising a lipid of plant origin and,
(b) lowering the temperature of the mixture sufficiently to preserve the mammalian cells and tissues in a viable condition.

2. A method according to claim 1 wherein the lipid is a phospholipid.

3. A method according to claim 2 wherein the phospholipid is phosphatidyl-choline.

4. A method according to claim 3 wherein phosphatidyl-choline is derived from soybeans.

5. A method according to claim 1 wherein the temperature is lowered to no more than about +4° C.

6. A method according to claim 1 wherein the mixture is subject to freezing temperatures.

7. A method according to claim 6 wherein the mixture is subjected to freezing temperatures as low as about −196° C.

8. A method according to claim 4 wherein the soybean phosphatidyl-choline is combined with hyaluronan.

9. A method according to claim 4 wherein the phosphatidyl-choline of soybean origin is present in the mixture in an amount of at least 1 g/l of the mixture.

10. A method according to claim 9 wherein the extender is a material isolated from soybeans containing about 5% to 50% by weight phosphatidyl-choline.

11. A method according to claim 10 wherein the extender is a material isolated from soybeans containing about 15% by weight phosphatidyl-choline and is present in the mixture in a concentration of about 0.20 g to 20 g per 100 mls.

12. A method according to claim 11 wherein the extender is a material isolated from soybeans containing about 15% by weight phosphatidyl-choline and is present in the mixture in a concentration of about 0.730 g to 7.30 g per 100 mls.

13. A method according to claim 4 wherein the mammalian cells and tissues are stored in liquid form at temperatures of no higher than +4° C.

14. A method according to claim 4 wherein the mammalian cells and tissues are stored in solid frozen form at temperatures as low as −196° C.

15. A method according to claim 4 wherein the mammalian cells and tissues comprise mammalian semen.

16. A method according to claim 15 wherein the mammalian semen is bull semen.
